(19) United States
(12) Patent Application Publication
(10) Pub. No.: US 2019/0159693 A1
(43) Pub. Date: May 30, 2019

(54) DIRECT ELECTROCARDIOGRAPHY MONITORING FOR ATRIAL FIBRILLATION DETECTION

(71) Applicant: Edwards Lifesciences Corporation, Irvine, CA (US)

(72) Inventors: Robert S. Schwartz, Inver Grove Heights, MN (US); Stanton J. Rowe, Newport Coast, CA (US); Gregory Bak-Boychuk, San Clemente, CA (US)

(21) Appl. No.: 16/178,486

(22) Filed: Nov. 1, 2018

Related U.S. Application Data
(60) Provisional application No. 62/591,888, filed on Nov. 29, 2017.

(51) Int. Cl. A61B 5/042; A61B 5/00; A61N 1/05; A61B 5/046; A61N 1/365

(52) U.S. Cl. CPC A61B 5/0422; A61B 5/0006; A61N 1/0597; A61B 5/046; A61B 5/0031; A61N 1/36507; A61B 5/4836; A61B 5/686; A61B 5/746

(57) ABSTRACT

A direct-implantable electrocardiographic (ECG) probe device includes a biocompatible housing, a battery disposed within the housing, one or more electrodes including an ECG electrode configured to sense an electrical signal in tissue of an atrium of a heart, circuitry disposed at least partially within the housing and configured to generate an ECG signal and wirelessly transmit the ECG signal through a chest wall, and an attachment structure configured to facilitate the attachment of the ECG probe device to a surface of the atrium.

### RELATED APPLICATION

This application claims priority to U.S. Provisional Application No. 62/591,888, filed Nov. 29, 2017, and entitled DIRECT ELECTROCARDIOGRAPHY MONITORING FOR ATRIAL FIBRILLATION DETECTION, the disclosure of which is hereby incorporated by reference in its entirety.

### BACKGROUND

### Field

The present disclosure generally relates to the field of medical surgery, such as cardiac surgery.

### Description of Related Art

Patients of cardiac surgery and other vascular operations can develop complications associated with fluid overload and/or atrial fibrillation post-operatively due to various conditions and/or factors. Atrial fibrillation is associated with certain health complications, including increased patient mortality, and therefore prevention and/or treatment of atrial fibrillation during surgery and/or post-operatively can improve patient health.

### SUMMARY

In some implementations, the present disclosure relates to a direct-implantable electrocardiographic (ECG) probe device comprising a biocompatible housing, a battery disposed within the housing, one or more electrodes including an ECG electrode configured to sense an electrical signal in tissue of an atrium of a heart, circuitry disposed at least partially within the housing and configured to generate an ECG signal and wirelessly transmit the ECG signal through a chest wall, and an attachment structure configured to facilitate the attachment of the ECG probe device to a surface of the atrium.

The attachment structure may comprise one or more suture holes. In certain embodiments, the attachment structure comprises a pin form configured to puncture the surface of the atrium. In certain embodiments, the direct-implantable ECG probe device further comprises a grounding structure. For example, the grounding structure may be disposed on an underside of the housing and configured to contact the surface of the atrium when the ECG probe device is implanted on the surface of the atrium.

The one or more electrodes may include a pacing electrode configured to introduce a jolt of electrical current to the surface of the atrium. In certain embodiments, the pacing electrode and the ECG electrode are the same. The housing may be at least partially disk-shaped.

In some implementations, the present disclosure relates to a heart monitoring system comprising a plurality of electrocardiographic (ECG) leads configured to be directly implanted in a surface of an atrium of a heart of a patient, sense an electrical signal in tissue of the atrium, and provide an ECG signal based on the sensed electrical signal. The heart monitoring system further comprises a monitor device coupled to the ECG leads and configured to receive the ECG signal, and a grounding pad electrically coupled to the monitor device.

In certain embodiments, the monitor device is configured to identify a P wave characteristic in the ECG signal associated with atrial fibrillation. The monitor device may be further configured to generate an alarm notification based on said identification of the P wave characteristic. The heart monitoring system may further comprise a plurality of pacing leads configured to be directly implanted in the surface of the atrium, the plurality of pacing leads being coupled to the monitor device. For example, the monitor device may be configured to present an electrical charge on one or more of the pacing leads in response to the ECG signal.

In some implementations, the present disclosure relates to a method of generating an electrocardiographic (ECG) signal. The method comprises implanting one or more ECG probes on a surface of a heart of a patient and generating an ECG signal using the implanted one or more ECG probe devices.

The one or more ECG probes may be discrete implantable devices. The method may further comprise wirelessly receiving the ECG signal from the one or more ECG probes through a chest wall of the patient. In certain embodiments, the one or more ECG probes are wire leads. The method may further comprise disposing the wire leads in a chest-access channel in a chest of the patient.

In certain embodiments, the method further comprises implanting one or more pacing leads in the surface of the heart. The method may further comprise delivering a dose of electrical current to the heart using the one or more pacing leads. In certain embodiments, the method further comprises closing a chest cavity of the patient after said implanting the one or more ECG probes and before said generating the ECG signal. The method may further comprise identifying a characteristic in the ECG signal that is associated with atrial fibrillation. In some embodiments, the method further comprises determining an impedance associated with a portion of the heart based at least in part on the ECG signal.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 provides an example cross-sectional view of a human heart.

FIG. 2 illustrates an example cross-sectional representation of a heart experiencing atrial fibrillation.

FIGS. 3A-3F illustrate example electrical conduction circuits that may form in the atria of the heart in connection with atrial fibrillation, such as post-operative atrial fibrillation.

FIG. 4A illustrates an example cardiac electrical signal.

FIG. 4B illustrates an example cardiac electrical signal that may be associated with atrial fibrillation.

FIG. 5 illustrates an embodiment of a heart having disposed and/or implanted thereon one or more direct-measurement electrocardiographic (ECG) probes in accordance with one or more embodiments.

FIG. 6 illustrates a top and side perspective view of a direct-implant ECG probe in accordance with one or more embodiments.

FIG. 7 illustrates a bottom and side perspective view of the ECG probe device shown in FIG. 6 in accordance with one or more embodiments.

FIG. 8 illustrates an exploded view of the ECG probe device shown in FIGS. 6 and 7.

FIG. 9 illustrates an embodiment of a direct-measurement ECG system in accordance with one or more embodiments.

FIG. 10 illustrates a portion of a heart having disposed and/or implanted therein one or more direct-measurement ECG leads and/or atrial pacing leads in accordance with one or more embodiments.

FIG. 11 illustrates a portion of a heart having disposed and/or implanted therein one or more conductive leads in accordance with one or more embodiments.

FIGS. 12A-12C illustrate example waveforms in accordance with one or more embodiments.

FIG. 13 illustrates a portion of a heart having disposed and/or implanted therein in accordance with one or more embodiments.

FIG. 14 is a flow diagram illustrating a process for monitoring stretching in biological tissue in accordance with one or more embodiments.

FIG. 15 is a flow diagram illustrating a process for calibrating a tissue stretch monitoring system in accordance with one or more embodiments.

### DETAILED DESCRIPTION

The headings provided herein are for convenience only and do not necessarily affect the scope or meaning of the claimed invention.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Terminology

Certain standard anatomical terms of location are used herein to refer to the anatomy of animals, and namely humans, with respect to the preferred embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Furthermore, references may be made herein to certain anatomical planes, such as the sagittal plane, or median plane, or longitudinal plane, referring to a plane parallel to the sagittal suture, and/or other sagittal planes (i.e., parasagittal planes) parallel thereto. In addition, “frontal plane,” or “coronal plane,” may refer to an X-Y plane that is perpendicular to the ground when standing, which divides the body into back and front, or posterior and anterior, portions. Furthermore, a “transverse plane,” or “cross-sectional plane,” or horizontal plane, may refer to an X-Z plane that is parallel to the ground when standing, that divides the body in upper and lower portions, such as superior and inferior. A “longitudinal plane” may refer to any plane perpendicular to the transverse plane. Furthermore, various axes may be described, such as a longitudinal axis, which may refer to an axis that is directed towards head of a human in the cranial direction and/or directed towards inferior of a human in caudal direction. A left-right or horizontal axis, which may refer to an axis that is directed towards the left-hand side and/or right-hand side of a patient. An anteroposterior axis which may refer to an axis that is directed towards the belly of a human in the anterior direction and/or directed towards the back of a human in the posterior direction.

Overview

In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The contraction of the various heart muscles may be prompted by signals generated by the electrical system of the heart, which is discussed in detail below. Certain embodiments disclosed herein relate to conditions of the heart, such as atrial fibrillation and/or complications or solutions associated therewith. However, embodiments of the present disclosure relate more generally to any health complications relating to fluid overload in a patient, such as may result post-operatively after any surgery involving fluid supplementation. That is, detection of atrial stretching as described herein may be implemented to detect/determine a fluid-overload condition, which may direct treatment or compensatory action relating to atrial fibrillation and/or any other condition caused at least in part by fluid overloading.

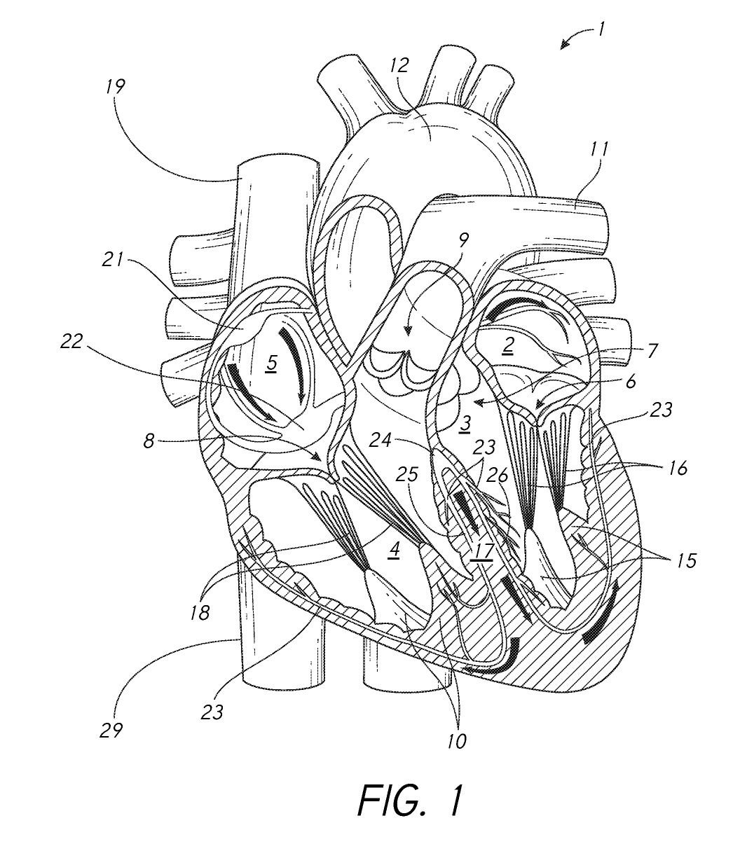

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (i.e., mitral and tricuspid) heart valves may further comprise a collection of chordae tendineae (16, 18) and papillary muscles (10, 15) for securing the leaflets of the respective valves to promote and/or facilitate proper coaptation of the valve leaflets and prevent prolapse thereof. The papillary muscles (10, 15), for example, may generally comprise finger-like projections from the ventricle wall. With respect to the mitral valve 6, a normal mitral valve may comprise two leaflets (anterior and posterior) and two corresponding papillary muscles 15. When the left ventricle 3 contracts, the intraventricular pressure forces the valve to close, while the chordae tendineae 16 keep the leaflets coapting together and prevent the valve from opening in the wrong direction, thereby preventing blood to flow back to the left atrium 2. With respect to the tricuspid valve 8, the normal tricuspid valve may comprise three leaflets (two shown in FIG. 1) and three corresponding papillary muscles 10 (two shown in FIG. 1). The leaflets of the tricuspid valve may be referred to as the anterior, posterior and septal leaflets, respectively. The valve leaflets are connected to the papillary muscles by the chordae tendineae 17, which are disposed in the right ventricle 4 along with the papillary muscles 10. The right ventricular papillary muscles 10 originate in the right ventricle wall, and attach to the anterior, posterior and septal leaflets of the tricuspid valve, respectively, via the chordae tendineae 17.

### Fluid Overload

Fluid overload or volume overload, which is referred to as hypervolemia, is a medical condition in which the vasculature contains too much fluid. Fluid-overload conditions can arise in connection with various types of surgical operations, including cardiac surgery. For example, fluid management through fluid infusion may be necessary or desirable in order to maintain adequate cardiac output, systemic blood pressure, and/or renal perfusion during or in connection with a surgical operation. Example settings in which fluid overload may develop include the administration of excessive fluid and sodium due to intravenous (IV) or fluids during surgical operations, such as atrial fibrillation ablation, valve repair or replacement, or other cardio/thoracic procedures, or fluid remobilization procedures associated with burn or trauma treatment.

Fluid overload can correlate with mortality in certain categories of patients. In order to restore or maintain desired fluid levels, it may be necessary or desirable to determine present volume status. According to some practices, fluid overload recognition and assessment involves strict documentation of fluid intakes and outputs. However, accuracy is fluid intake/output tracking can be difficult to achieve over time, and there are a wide variety of methods utilized to evaluate, review, and utilize fluid tracking data. Furthermore, errors in volume status determination can result in a lack of essential treatment or unnecessary fluid administration, either of which can present serious health risks.

As described herein, fluid overload associated with fluid administration of fluid in association with a surgical operation can result in post-operative onset of atrial fibrillation. Furthermore, fluid overload conditions can cause or be associated with various other conditions, including pulmonary edema, cardiac failure, delayed recovery, tissue breakdown, and/or at least partially impaired function of bowels or other organs. Therefore, the evaluation of volume status can be important before, during, and/or after a surgical operation, such as cardia surgery. Once identified, fluid overload may be treated in a variety of ways, including cessation or reduction of fluid administration, administration of diuretics, and/or fluid/letting.

For at least the reasons outlined above, determination/detection of fluid overload conditions can be critical or important to prevention or treatment of various adverse health conditions. However, the lack of available volume overload sensors that conveniently and accurately measure or indicate fluid overload can be problematic. Embodiments of the present disclosure provide improved systems, devices, and methods for determining/detecting a fluid overload condition by monitoring tissue stretching in fluid-containing organs or tissue. For example, tissue stretching in an atrium (or ventricle) of a hear, as described in detail herein, can indicate a fluid overload, or impending fluid overload, condition. The embodiments of the present disclosure advantageously provide removable devices/systems for measuring tissue stretching associated with fluid overload in a relatively convenient manner compared to pressure measurement fluid tracking using, for example, peripherally-inserted central catheter (PICC or PIC line), or other known mechanism for tracking of fluid pressure or other characteristic(s). Certain embodiments of the present disclosure provide improvements over other patient monitoring solutions by providing systems, devices, and methods for directly measuring organ or tissue stretching, wherein it is not necessary to infer tissue stretching from echo or x-ray imaging. Direct tissue-measuring in accordance with embodiments of the present disclosure may be used to measure atrial tissue stretching, or stretching of other organs or tissue, including but not limited to gestational stretch measurement of uterine tissue or other pregnancy-related stretching, prostate stretching/enlargement, liver tissue stretching, colon stretching/enlargement, or other tissue/organ.

### Cardiac Electrical System

The electrical system of the heart generally controls the events associated with the pumping of blood by the heart. With further reference to FIG. 1, the heart 1 comprises different types of cells, namely cardiac muscle cells (also known as cardiomyocytes or myocardiocytes) and cardiac pacemaker cells. For example, the atria (2, 5) and ventricles (3, 4) comprise cardiomyocytes, which are the muscle cells that make up the cardiac muscle. The cardiac muscle cells are generally configured to shorten and lengthen their fibers and provide desirable elasticity to allow for stretching. Each myocardial cell contains myofibrils, which are specialized organelles consisting of long chains of sarcomeres, the fundamental contractile units of muscle cells.

The electrical system of the heart utilizes the cardiac pacemaker cells, which are generally configured to carry electrical impulses that drive the beating of the heart 1. The cardiac pacemaker cells serve to generate and send out electrical impulses, and to transfer electrical impulses cell-to-cell along electrical conduction paths. The cardiac pacemaker cells further may also receive and respond to electrical impulses from the brain. The cells of the heart are connected by cellular bridges, which comprise relatively porous junctions called intercalated discs that form junctions between the cells. The cellular bridges permit sodium, potassium and calcium to easily diffuse from cell-to-cell, allowing for depolarization and repolarization in the myocardium such that the heart muscle can act as a single coordinated unit.

The electrical system of the heart comprises the sinoatrial (SA) node 21, which is located in the right atrium 5 of the heart 1, the atrioventricular (AV) node 22, which is located on the interatrial septum in proximity to the tricuspid valve 8, and the His-Purkinje system 23, which is located along the walls of the left 3 and right 4 ventricles.

A heartbeat represents a single cycle in which the heart's chambers relax and contract to pump blood. As described above, this cycle includes the opening and closing of the inlet and outlet valves of the right and left ventricles of the heart. Each beat of the heart is generally set in motion by an electrical signal generated and propagated by the heart's electrical system. In a normal, healthy heart, each beat begins with a signal from the SA node 21. This signal is generated as the vena cavae (19, 29) fill the right atrium 5 with blood, and spreads across the cells of the right 5 and left 2 atria. The flow of electrical signals is represented by the illustrated shaded arrows in FIG. 1. The electrical signal from the SA node 21 causes the atria to contract, which pushes blood through the open mitral 6 and tricuspid 8 valves from the atria into the left 3 and right 4 ventricles, respectively.

The electrical signal arrives at the AV node 22 near the ventricles, where it may slow for an instant to allow the right 4 and left 3 ventricles to fill with blood. The signal is then released and moves along a pathway called the bundle of His 24, which is located in the walls of the ventricles. From the bundle of His 24, the signal fibers divide into left 26 and right 25 bundle branches through the Purkinje fibers 23. These fibers connect directly to the cells in the walls of the left 3 and right 4 ventricles. The electrical signal spreads across the cells of the ventricle walls, causing both ventricles to contract. Generally, the left ventricle may contract an instant before the right ventricle. Contraction of the right ventricle 4 pushes blood through the pulmonary valve 9 to the lungs (not shown), while contraction of the left ventricle 3 pushes blood through the aortic valve 6 to the rest of the body. As the electrical signal passes, the walls of the ventricles relax and await the next signal.

### Atrial Fibrillation

FIG. 1, as described above, illustrates a normal electrical flow, resulting in a regular heart rhythm, that may be associated with a generally healthy heart. However, in certain patients or individuals, various conditions and/or events can result in compromised electrical flow, causing the development and/or occurrence of an abnormal heart rhythm. For example, atrial fibrillation is a condition associated with abnormal electrical flow and/or heart rhythm characterized by relatively rapid and irregular beating of the atria.

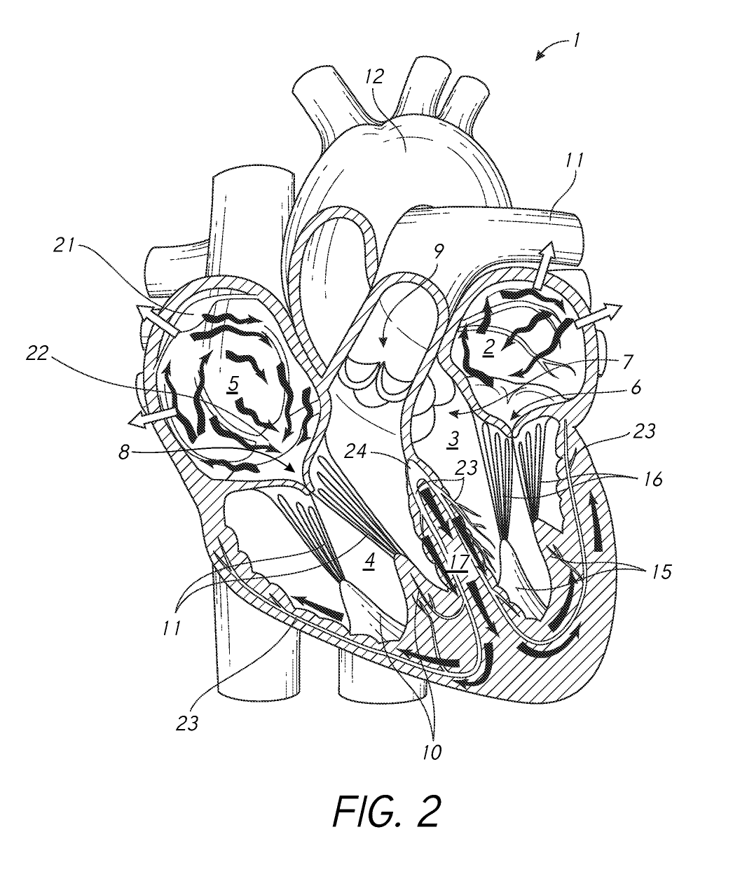

FIG. 2 illustrates an example cross-sectional representation of the heart 1 of FIG. 1 experiencing atrial fibrillation. When atrial fibrillation occurs, the normal regular electrical impulses generated by the sinoatrial (SA) node 21 in the right atrium 5 may become overwhelmed by disorganized electrical impulses, which may lead to irregular conduction of ventricular impulses that generate the heartbeat. The illustrated shaded arrows represent the erratic electrical impulses that can be associated with atrial fibrillation. Atrial fibrillation generally originates in the right atrium 5, that where conduction path disturbances begin.

Various pathologic developments can lead to, or be associated with, atrial fibrillation. For example, progressive fibrosis of the atria may contribute at least in part to atrial fibrillation. The formation of fibrous tissue associated with fibrosis can disrupt or otherwise affect the electrical pathways of the cardiac electrical system due to interstitial expansion associated with tissue fibrosis. In addition to fibrosis in the muscle mass of the atria, fibrosis may also occur in the sinoatrial node 21 and/or atrioventricular node 22, which may lead to atrial fibrillation.

Fibrosis of the atria may be due to atrial dilation, or stretch, in some cases. Dilation of the atria can be due to a rise in the pressure within the heart, which may be caused by fluid overload, or may be due to a structural abnormality in the heart, such as valvular heart disease (e.g., mitral stenosis, mitral regurgitation, tricuspid regurgitation), hypertension, congestive heart failure, or other condition. Dilation of the atria can lead to the activation of the renin aldosterone angiotensin system (RAAS), and subsequent increase in matrix metalloproteinases and disintegrin, which can lead to atrial remodeling and fibrosis and/or loss of atrial muscle mass.

In addition to atrial dilation, inflammation in the heart can cause fibrosis of the atria. For example, inflammation may be due to injury associated with a cardiac surgery, such as a valve repair operation, or the like. Alternatively, inflammation may be caused by sarcoidosis, autoimmune disorders, or other condition. Other cardiovascular factors that may be associated with the development of atrial fibrillation include high blood pressure, coronary artery disease, mitral stenosis (e.g., due to rheumatic heart disease or mitral valve prolapse), mitral regurgitation, hypertrophic cardiomyopathy (HCM), pericarditis, and congenital heart disease. Additionally, lung diseases (such as pneumonia, lung cancer, pulmonary embolism, and sarcoidosis) may contribute to the development of atrial fibrillation in some patients.

### Development of Post-Operative Atrial Fibrillation

In addition to the various physiological conditions described above that may contribute to atrial fibrillation, in some situations, atrial fibrillation may be developed in connection with a vascular operation, such post-operatively in the days following a vascular operation. Various factors may bear on the likelihood of a patient developing post-operative atrial fibrillation, such as age, medical history (e.g., history of atrial fibrillation, chronic obstructive pulmonary disease (COPD)), concurrent valve surgery, withdrawal of post-operative treatment (e.g., beta-adrenergic blocking agents (i.e., beta blocker), angiotensin converting enzyme inhibitors (ACE inhibitor)), beta-blocker treatment (e.g., pre-operative and/or post-operative), ACE inhibitor treatment (e.g., pre-operative and/or post-operative), and/or other factors. Generally, for patients that experience post-operative atrial fibrillation, the onset of atrial fibrillation may occur approximately 2-3 days after surgery.

Atrial dilation/stretching may be considered a primary variable associated with post-operative atrial fibrillation. In some situations, occurrence of post-operative atrial fibrillation may follow, at least in part, the following progression: First, the patient undergoes a surgical procedure, such as a vascular surgical operation (e.g., cardiac surgery). In connection with the operation, the patient may be subject to drug and/or fluid management. For example, the patient may receive post-surgery intravenous (IV) fluid loading and/or diuretic/drug volume management. Such treatment may result in fluid overload, which may lead to atrial stretching due to increased pressure in one or more atria. Atrial stretching may occur over a 1-2 day period, or longer, resulting in dilation of one or both of the atria. Fibrotic atrial tissue may form in connection with atrial stretching. Atrial stretching and/or fibrotic atrial tissue formation may result in an increased incidence of post-operative atrial fibrillation (e.g., 30-40% increased incidence of post-operative atrial fibrillation). In addition, inflammation associated with surgical operations can contribute the onset of post-operative atrial fibrillation, and reduced inflammation may generally correlate to a reduced risk of atrial fibrillation.

Post-operative atrial fibrillation is generally associated with increased patient morbidity, as well as economic burden. For example, post-operative atrial fibrillation is generally associated with increased incidence of congestive heart failure, increased hemodynamic instability, increase renal insufficiency, increased repeat hospitalizations, increased risk of stroke, and increase in hospital mortality and 6-month mortality. Post-operative atrial fibrillation also represents a systemic burden, wherein intensive care unit (ICU) stay, hospital length of stay, hospital charges, and rates of discharge to extended care facilities are increased as a result of post-operative atrial fibrillation.

Furthermore, because an initial incidence of atrial fibrillation generally results in recurring, progressively more severe, episodes of atrial fibrillation in a patient, the consequences of allowing atrial fibrillation to develop post-operatively can be considered particularly severe for a given patient. For example, a given patient may initially experience intermittent/sporadic episodes of atrial fibrillation as a result of post-operative atrial dilation and/or inflammation, with recurring episodes progressively increasing in frequency and/or severity.

### Direct Electrocardiography Monitoring

Electrocardiographic (ECG) measurements can provide readings of electrical activity in the heart. For example, as described above, the beating of the heart is generally driven by signals generated in the sinoatrial node and passed through the atria along conduction pathways and into the ventricles of the heart. In addition to providing various other indicators of physiological health and/or conditions, ECG measurements may be indicative of atrial fibrillation in some situations.

ECG readings may be obtained through the placement of ECG leads, which are often affixed to the external chest wall of the patient in proximity to the heart. The leads placed on the surface of the chest may pick up electrical signals generated in the heart and provide a reading reflective thereof, which may be analyzed or used for various purposes. However, the electrical resistance of the chest wall and distance between the outer surface of the chest and the electrical nodes of the heart may result in ECG signals that are not desirably strong/clear and/or require filtering in order to determine or provide suitable electrical signal information. That is, ECG readings acquired using externally-placed leads may not provide sufficient sensitivity for interpreting the electrical signals of the heart with respect to certain conditions, such as atrial fibrillation, or other conditions. or the potential early detection of volume overload

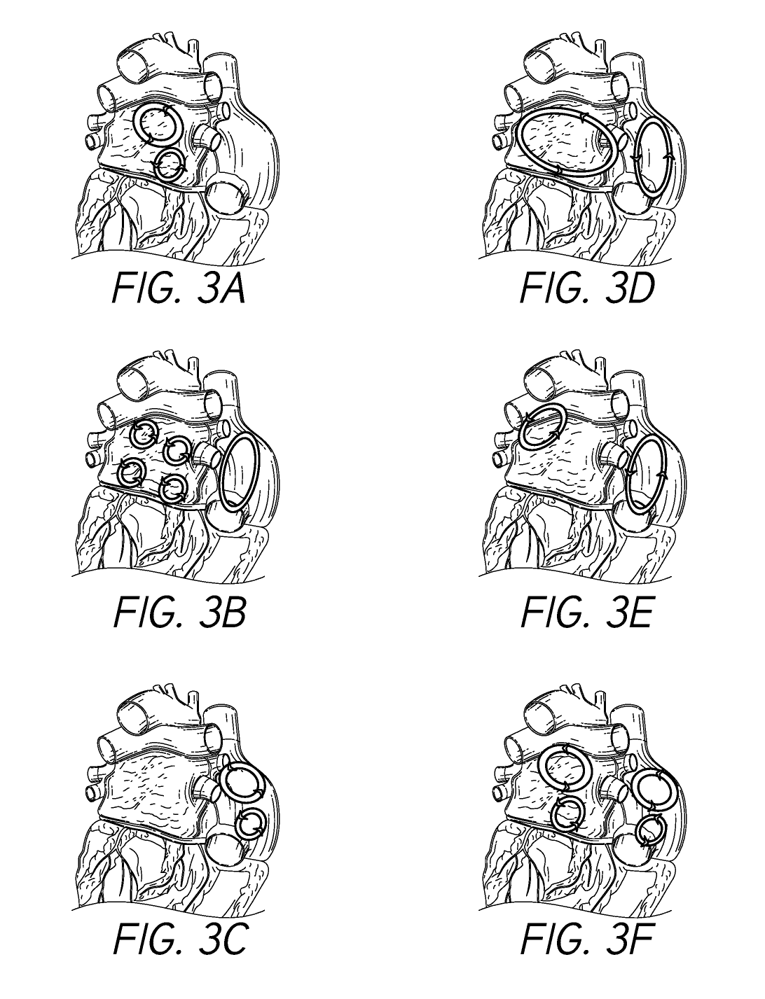

The presence of atrial fibrillation may generally be characterized by disturbance(s) in electrical conduction paths in the atria of the heart, and in particular in the right atrium. FIGS. 3A-3F illustrate example electrical conduction circuits that may form in the atria of the heart in connection with atrial fibrillation, such as post-operative atrial fibrillation. For example, as shown, circular conduction paths/circuits may form in connection with atrial fibrillation. Such paths/circuits may not be measurable using ECG leads disposed on the patient's chest wall.

Certain embodiments disclosed herein relate to methods and devices/probes that may be placed directly onto the atrial surface to measure discrete changes in voltage signals associated with atrial stretching and/or atrial fibrillation. For example, open-chest surgical procedures may provide an opportunity to a physician/technician to implant such electrical probes directly onto the atrial surface. Although atrial stretching is described in detail in connection with certain embodiments disclosed herein, it should be understood that such embodiments may be applicable to tissue-stretching detection/measurement with respect to other types of organs or tissue, or even to other types of materials in non-biological applications.

In addition to electrical probe functionality, implants in accordance to embodiments of the present disclosure may further be implemented to provide electrical pacing for the atria and/or other portions of the heart, as described in detail below. The term “pacing” is used herein according to its broad and ordinary meaning, and may refer to the generation and/or provision of electrical impulses to signals that are delivered by electrodes to promote contraction of one or more muscles of the heart and/or at least partially regulate the electrical conduction system of the heart, or any other generation, provisions, and/or introduction of electrical signals into biological tissue of the heart or other organ or tissue. Furthermore, it should be understood that discussion herein of ECG electrodes, ECG leads, conductive leads, ECG probes, or variations thereof or the like do not necessarily refer to external ECG electrode pads designed for placement on a patient's chest or other external skin area, but rather generally refer to devices or elements directly implanted on/in an internal organ of a patient. In some embodiments, the present disclosure provides a battery-powered probe device that may at least partially pierce the outer tissue/surface of one or more atria of the heart to monitor electrical signals of the heart. The direct-implant electrical measurement probes may be removable in some embodiments and may further provide defibrillation capabilities. Electrical measurement probes in accordance with the present disclosure may provide filtered ECG voltage signals and may be used to sense discrete electrical changes that may be associated with the onset of atrial fibrillation.

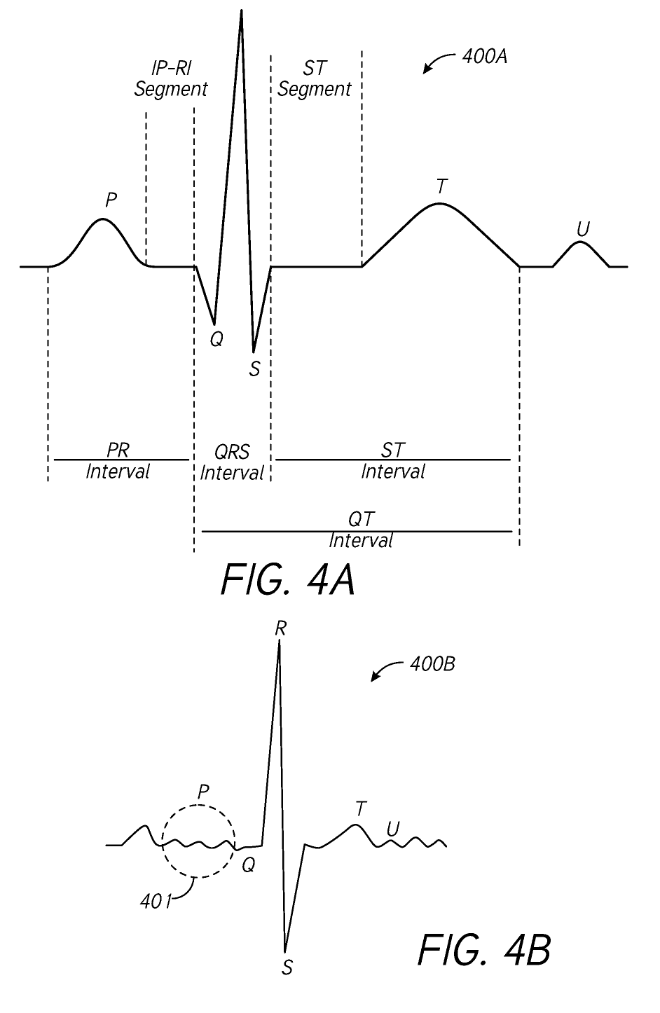

Electrical conduction path disturbances in the heart, such as disturbed electrical conduction paths similar to those illustrated in FIGS. 3A-3F, may be determined or measured in various ways. For example, interatrial conduction path disturbances may be determined through analysis of ECG signals, and in particular, P wave signals. For example, FIG. 4A illustrates an example ECG signal 400A, which may be generally associated with a cardiac electrical signal of a healthy patient. The electrical signal 400A comprises various components or features, which may be associated with different conditions or factors related to the electrical impulse of the heart. For example, as denoted in the diagram of FIG. 4A, the signal 400A includes a P wave, which represents the depolarization of the atria. For example, atrial depolarization generally spreads from the sinoatrial (SA) node towards the atrioventricular (AV) node, and generally from the right atrium to the left atrium. As described in detail below, the shape and/or features of the P wave may be indicative of atrial fibrillation and/or the onset thereof.

In addition to the P wave, the signal 400A further comprises a PR interval, which may generally be measured from the beginning of the P wave to the beginning of what is referred to as the QRS interval. The PR interval may generally reflect the time an electrical pulse takes to travel from the SA node through the AV node. The illustrated PR segment represents the portion of the signal 400A after the P wave and before the QRS interval. The QRS interval may represent a relatively rapid depolarization of the right and left ventricles, which may be associated with the discharging of blood from the ventricles as the muscle mass of the ventricles contracts. The signal 400A further illustrates an ST segment, which connects the QRS complex to another wave, referred to as the T wave. The ST segment may generally represent the period when the ventricles are depolarized. The T wave represents the repolarization of the ventricles. The signal 400A further includes a U wave, which may be associated with the repolarization of the interventricular septum. Further, the QT interval may be measured from the beginning of the QRS complex to the end of the T wave.

Generally, there may be a relatively strong correlation between interatrial conduction disturbances and post-operative atrial fibrillation. Such relationship is discussed in “Interatrial Conduction Disturbances in Postoperative Atrial Fibrillation: A Comparative Study of Pre-wave Dispersion and Doppler Myocardial Imaging in Cardiac Surgery.” Hatam et al., Journal of Cardiothoracic Surgery (2014), which is incorporated by reference herein.

FIG. 4B illustrates a cardiac electrical signal 400B that may be associated with atrial fibrillation. The signal 400B shown in FIG. 4B may generally include certain P wave dispersions 401, which may be caused at least in part by electrical conduction path disturbances, such as those illustrated in FIGS. 3A-3F, described above. Therefore, atrial fibrillation, such as post-operative atrial fibrillation, may generally be recognizable through analysis of a sufficiently clean ECG signal, and in particular, the P wave thereof. Generally, the shape and/or duration of the P wave may be an indicator of atrial fibrillation, or future onset of atrial fibrillation. The duration of P wave dispersions may be associated with the onset of post-operative atrial fibrillation. Therefore, it may be desirable to measure P wave dispersions in order to institute responsive action to prevent atrial fibrillation. In some patients, P wave dispersions of approximately 15-20 ms may be associated with post-operative atrial fibrillation.

Due to the signal quality generally associated with ECG signals generated using ECG leads placed on external chest surfaces, it may be desirable to place ECG leads in positions in more close or direct proximity to the source of the electrical signals of the heart. Certain embodiments disclosed herein provide methods for generating ECG signals and/or determining the presence or susceptibility of atrial fibrillation using devices/probes that can be placed directly onto the atrial surface. For example, access to the atrial surface may be available to a physician/technician in connection with an open-chest surgical procedure. Such methods and devices may be used to measure discrete changes in voltage signals associated with atrial stretching, which can be a cause of, and/or associated with, atrial fibrillation, as described above. Direct placement of ECG leads onto atrial walls can provide relatively more direct voltage measurement. For example, atrial tissue stretching can cause local conduction path disturbances to the atrial voltage signal, which may take the form of circular conduction paths, as described above. Direct placement of ECG leads/probes, which may take the form of thumbtack-shaped buttons in some embodiments, may provide relatively more sensitive measurements of voltage disturbances caused by atrial stretching. With more sensitive voltage measurement devices, the stretching of atrial tissue may be more quickly and/or easily detectable, and therefore prevention and/or treatment of atrial fibrillation may be more effective in connection with the embodiments disclosed herein.

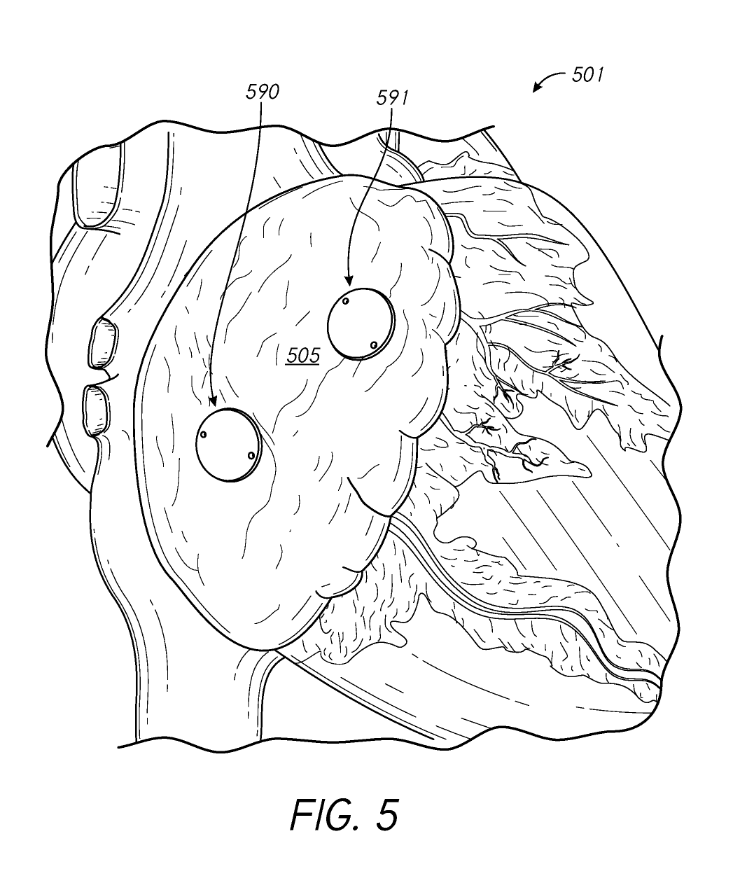

FIG. 5 illustrates an embodiment of a heart 501 having disposed and/or implanted thereon one or more direct-measurement ECG probes 590, 591. In certain embodiments, the ECG measurement probes 590, 591 may be directly placed onto the atrial wall, and may be configured to provide discrete measurements of disturbances to the atrial voltage signal or path. In certain embodiments, the ECG measurement probes 590, 591 may be sutured or otherwise attached to the atrium wall and may be configured to locally measure the ECG signal. Although the ECG measurement devices 590, 591 are illustrated as implanted on the right atrium, it should be understood that such devices may be implanted on any surface of the heart, such as on the surface of the left atrium and/or surfaces of the ventricles. Signals generated by the measurement probes 590, 591 may be filtered and/or analyzed in order to identify electrical conduction path disturbances, which may be associated with atrial fibrillation. That is, the devices 590, 591 may be positioned and/or configured to provide information indicative of circular conduction paths, as described herein. Although ECG signals generated using devices directly implanted or disposed on surfaces of the heart in accordance with the present disclosure are described herein as being used for atrial fibrillation detection and/or treatment, it should be understood that ECG information generated by direct-attachment ECG measurement probes in accordance with the present disclosure may be used for any suitable or desirable purposes.

Although FIG. 5 illustrates circular “thumbtack”-type direct-measurement probes, it should be understood that direct-measurement probes in accordance with the present disclosure may have any suitable or desirable shape or form. In some implementations, the devices 590, 591 may be placed at or proximate to the normal electrical conduction paths generally associated with the right atrium, or other region of the heart. Although two measurement devices 590, 591 illustrated, it should be understood that in some implementations, a single measurement device is implanted/used. Furthermore, more than two measurement devices may be used in some embodiments.

The direct placement of ECG measurement probes as shown in FIG. 5 may allow for discrete measurement of conduction path disturbances at the source of the cardiac electrical signals, which may provide relatively better electrical clarity through direct contact with the atrium. Such improved electrical clarity may allow for early detection of atrial fibrillation onset, such as post-operatively. In some implementations, ECG probes in accordance with the present disclosure may be implanted directly onto one or more ventricles of the heart in order to detect conditions other than atrial fibrillation.

The ECG probes 590, 591 shown in FIG. 5, in addition, or as an alternative, to the ECG signal detection and measurement described above, may be configured to provide electrical pacing functionality. For example, where atrial fibrillation is detected or predicted, the devices may be configured to provide an electrical jolt, or dose of electric current, to correct the cardiac rhythm. For example, the jolt of electrical current may serve to depolarize at least a portion of the heart and allow the sinoatrial node to re-establish normal electrical conduction paths. For example, one or more jolts of electrical current from the devices 590, 591 may cause blood to be squeezed out of the atrium and/or ventricles and allow for rebalancing of fluid distribution. Electrical jolts may be powered using an internal battery of the ECG device, or a conductive lead. Such battery may be configured to last a temporary duration during which post-operative atrial fibrillation may be experienced, such as up to five days or more. In some embodiments, the electrical jolts may trigger an alarm or other indicator, which may occur substantially automatically. Such alarm/indicator may be interpreted by an operator, such as a physician or nurse, wherein responsive action may be taken in response thereto, such as adjustment to fluid management for the patient. The pacing functionality of the devices 590, 591 may help to prevent further scar tissue formation and/or break down of electrical conduction paths.

In some implementations, the direct-implant ECG probes 590, 591 may be configured and/or designed to be permanently implanted in the tissue of the atrium. Therefore, such implantation may make certain activities dangerous or undesirable, such as magnetic resonance imaging (MRI), or other magnetism-based procedures. Furthermore, where the implanted devices generate jolts of electrical current as described above, such current may cause a disturbance to electrical signals read by external chest-applied ECG monitor leads.

It may be desirable for the monitoring of atrial voltage signal disturbances corresponding with atrial stretch, as performed using direct-implant ECG probes in accordance with the present disclosure, to be communicated to physicians or other operators so that treatment modifications may be administered in response to the measured ECG signals. For example, where atrial fibrillation is detected or predicted, the reduction of intravenous (IV) fluids may be desirable to prevent further stretching of the atrial tissue.

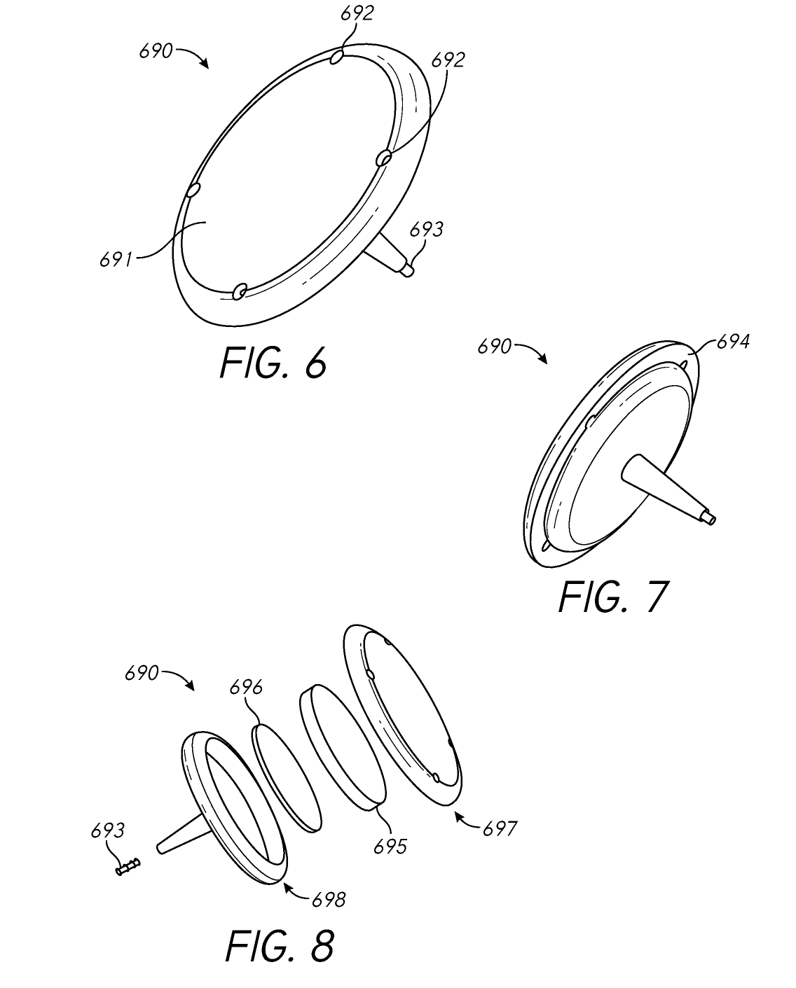

FIGS. 6-8 illustrate different views of an example direct-implant ECG probe in accordance with one or more embodiments. FIG. 6 illustrates a top and side perspective view of an ECG probe device 690. The ECG probe 690 may be similar in certain respects to one or more of the probes 590, 591 illustrated in FIG. 5 and described above. The probe device 690 may comprise one or more suture holes 692, which may be used to suture the device and/or otherwise secure or attach the device to the surface of an atrium or other region of the heart. For example, the probe device 690 may be configured to be sutured to the right atrium of the heart, such as on, or proximate to, electrical conduction pathways of the atrium. In certain embodiments, the ECG probe device 690 comprises a metallic sensing electrode and/or pacing lead 693, which may be configured to at least partially puncture the atrial tissue. The lead 693 may be used to measure electrical signals in the heart tissue. Furthermore, in some embodiments, the lead 693 and/or other component of the device 690, may be configured to provide a pacing jolt of electrical current, as described above. One or more components of the device 690 may be contained within a housing 691, such as a plastic or other encapsulating form, which may comprise one or more components fitted together to collectively form the housing 691.

FIG. 7 illustrates a bottom and side perspective view of the ECG probe device 690 shown in FIG. 6. In certain embodiments, the ECG probe device 690 may comprise an electrical grounding structure or form, which may comprise electrically conductive material, such as metal or the like. For example, the grounding structure may take the form of a ring electrode 694, which may be disposed at least partially on an underside of the ECG device 690 and may contact the atrial tissue when the device 690 is implanted thereon.

FIG. 8 illustrates an exploded view of the ECG probe device 690 shown in FIGS. 6 and 7. FIG. 8 illustrates various internal components that may be incorporated in the device 690. For example, certain internal components may be contained within the housing 691 of the ECG probe device 690, which may comprise a top portion 697 and a bottom portion 698 in some embodiments. The top 697 and bottom 698 portions may be configured to be mated together to collectively provide an enclosure for the internal components. Any suitable or desirable internal components may be contained within the ECG probe device 690. For example, a battery 695 may be included, which may provide electrical power that may be used to provide electrical pacing current through the pacing lead 693 in certain embodiments. In some embodiments, the battery 695 may have a lifespan of up to 10 days or more. Additionally or alternatively, the internal components of the ECG device 690 may comprise a circuit board 696, which may incorporate certain devices, traces, and/or other electrical components, which may be used to implement any of the functionality disclosed herein. For example, in some implementations, the probe device 690 is configured to implement wireless data and/or power transmission and/or reception. Such wireless transceiver components may be incorporated in the circuit board 696 or other circuitry of the device 690.

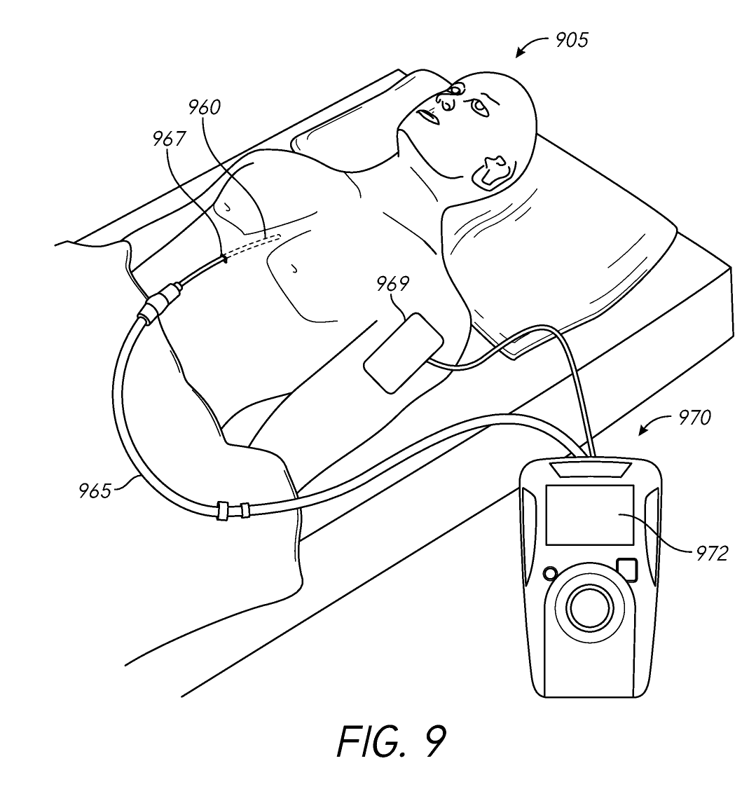

FIG. 9 illustrates an embodiment of a direct-measurement ECG system 900 in accordance with one or more embodiments of the present disclosure. While FIGS. 5-8, as described above, relate to discrete direct-implantable ECG probes/devices for measuring ECG signals and/or providing electrical current for pacing of the heart, the system 900 of FIG. 9 incorporates implantable conductive leads, which may be directly implanted into the right atrium or other region of the heart for ECG monitoring and/or pacing. Placement of direct ECG leads into the atria of the heart may allow for detection of atrial stretch and/or electrical conduction path disturbances, as described herein. In certain embodiments, the ECG leads 960 may be placed or anchored in the atrial wall. The leads 960 may further be passed through the chest wall at a chest access point 967, or through a chest drainage tube or other access point, and may be pulled out of the chest through the chest access 167, such as at the time of patient discharge. The leads 960 may comprise ECG measurement leads and/or pacing leads, either of which may be retrievable through a chest tube or through a skin access point. In certain embodiments, the leads 960 comprise two ECG measurement wires and/or two pacing wires.

Unlike permanent direct-implanted ECG probes/devices as described above, the direct attachment ECG leads 960 may advantageously be fully removed from the chest cavity of the patient 905, such that no conductive implant is left behind in the chest cavity of the patient. The removability feature(s) of the ECG device advantageously provide a convenient mechanism for providing pacing, ECG measurement, and/or tissue stretching measurement functionality, while not requiring permanent implants or prolonged maintenance of implanted device(s) in the body, which can improve long-term health prospects compared to permanent or indefinite/long-term implant devices.

The system 900 may further comprise a monitor unit 970. In certain embodiments, the monitor unit 970 may provide a low-filter ECG monitor with alarm notification functionality. For example, the monitor 970 may receive the ECG signal from ECG leads 960 and trigger an alarm or other notification or information display in response to the detected ECG signal. The monitor 970 may incorporate one or more light sources, which may provide an alarm or notification. Alternatively or additionally, the monitor 970 may comprise one or more other audio or visual components for providing alarm notifications. The monitor 970 may alarm or notify a physician or technician of early detection of atrial fibrillation, such that responsive or preventative measures may be implemented. The system 900 may further comprise an electrical ground structure or component 969, such as an adhesive ground pad or the like.

The monitor unit 970 may analyze the ECG waveform and identify changes in the waveform. For example, the monitor 970 may be configured to identify a difference in time (e.g. milliseconds) between receipt of an electrical signal at a first ECG lead and at a second ECG lead of the leads 960. For example, during a period of time after surgery, an increase in time of appearance of electrical signals at a first lead relative to a second lead may indicate atrial stretch. Furthermore, if an electrical signal that is sensed at a first lead is not sensed at a second lead, such condition may indicate a breakdown or disturbance in the electrical conduction path, which may be associated with atrial fibrillation. In some implementations, the monitor 970 may be configured to measure the electrical resistance between two direct-implanted ECG leads. An increase in electrical resistance between attachment points of an atrium may indicate increased distance, and/or formation of scar tissue, due to atrial stretching. Therefore, where electrical resistance changes and/or electrical disturbances are observed, such condition may be interpreted as an indication that the patient is falling into atrial fibrillation.

The monitor 970 and/or system 900 may be configured with pacing capabilities, wherein the leads 960 implanted in the chest cavity of the patient 905 may include one or more pacing leads. For example, in addition to ECG leads, a separate set of two or more pacing leads may be provided that are configured to provide dosages of electrical current to one or more regions of the heart, such as to the right atrium. The pacing leads may be accessed externally through a common access point 967, or may be accessible through a separate access point, such as through a separate channel through the chest wall, or through a chest drainage tube, or the like. The monitor 970 may be configured to execute pacing charges using the pacing leads. Such charges may be powered by the monitor, which may receive power from an external source.

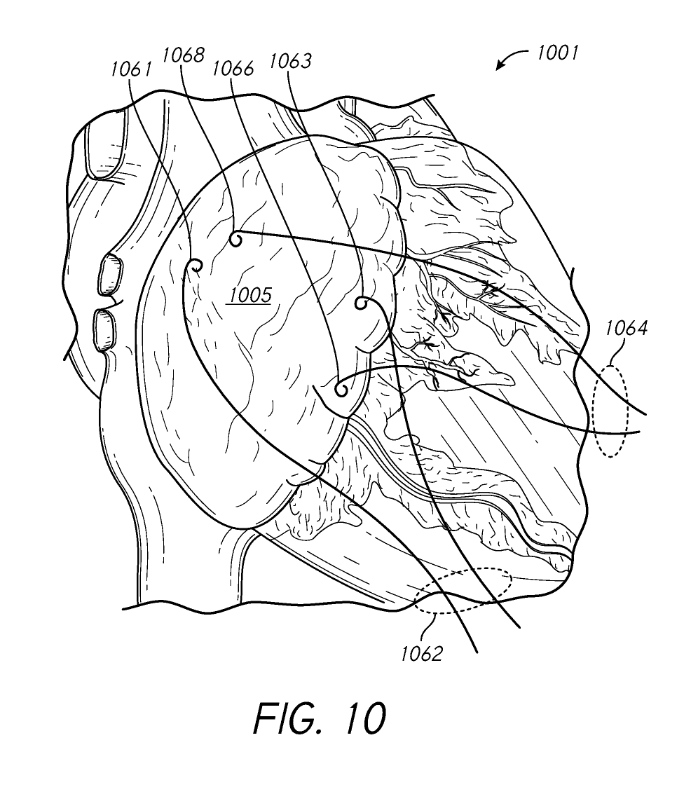

FIG. 10 illustrates an embodiment of a heart 1001 having disposed and/or implanted thereon one or more direct-measurement ECG leads 1062 and/or atrial pacing leads 1064. Although two ECG detection leads 1062 and two pacing leads 1064 are shown implanted in the image of FIG. 10, it should be understood that any number of ECG detection leads and/or pacing leads may be used in accordance with embodiments of the present disclosure.

The leads 1062, 1064 may have corkscrew-type anchoring distal ends, which may be twisted or pushed into the atrial tissue to puncture and anchor to the tissue. Although two ECG detection leads are shown, in some embodiments, a single lead may be used for ECG detection. For example, a single lead may be utilized to monitor the electrical conduction path and/or detect electrical disturbances. In embodiments having two or more ECG detection leads, such leads may be used to determine and/or analyze electrical flow from one point in the atrium to another, or from the atrium to another point or region of the heart. For example, the timing of when signals are received at first and second points associated with the first 1061 and second 1063 ECG detection leads may be analyzed to determine certain parameters.

The ECG leads 1062 and/or pacing leads 1064 may be removed from the heart by pulling from an externally accessible portion of such leads, which may thereby cause the anchor portions of the leads to straighten out and/or become dislodged from their anchored positions. The removability feature(s) of the ECG leads 1062 provide a convenient mechanism for providing pacing, ECG measurement, and/or tissue stretching measurement functionality, while not requiring permanent implants or prolonged maintenance of implanted device(s) in the body, which can improve long-term health prospects compared to permanent or indefinite/long-term implant devices.

The direct-implanted ECG leads 1062 may be used to generate ECG signals, which may be subject to modified signal filtering to sense discrete voltage signal disturbances. Because of the direct connection of the ECG leads 1064 to the atrium tissue, the resultant ECG signals generated thereby may advantageously be relatively clear compared to ECG signals generated by chest ECG leads. The pacing leads 1064 may be used to provide a jolt of electrical current to place the atrium back into proper cardiac rhythm once electrical disturbances are detected.

The present disclosure describes various means for measuring stretching, dilation, expansion, contraction, compression, shrinking and/or other modification of tissue or change in relative distance between two or more points or areas of tissue, such as atrial tissue. In some implementations, the present disclosure provides systems, devices, and methods for determining tissue stretching based on, or through analysis of, electrical signals or waveforms detected and/or transmitted in atrial tissue. Such signals/waveforms may be used to determine impedance and/or resistance of tissue between two or more points, wherein change in such impedance/resistance may indicate atrial stretch between the relevant points. Impedance and/or waveform/signal analysis or determination may be implemented using one or more direct-attached conductive leads on the atrium surface. The signals/waveforms analyzed using direct-attached conductive lead(s) may be natural cardiac electrical signals or may be introduced into the target tissue by one or more conductive leads or other devices. For example, a conductive lead may be used to introduce a test signal for waveform/impedance analysis.

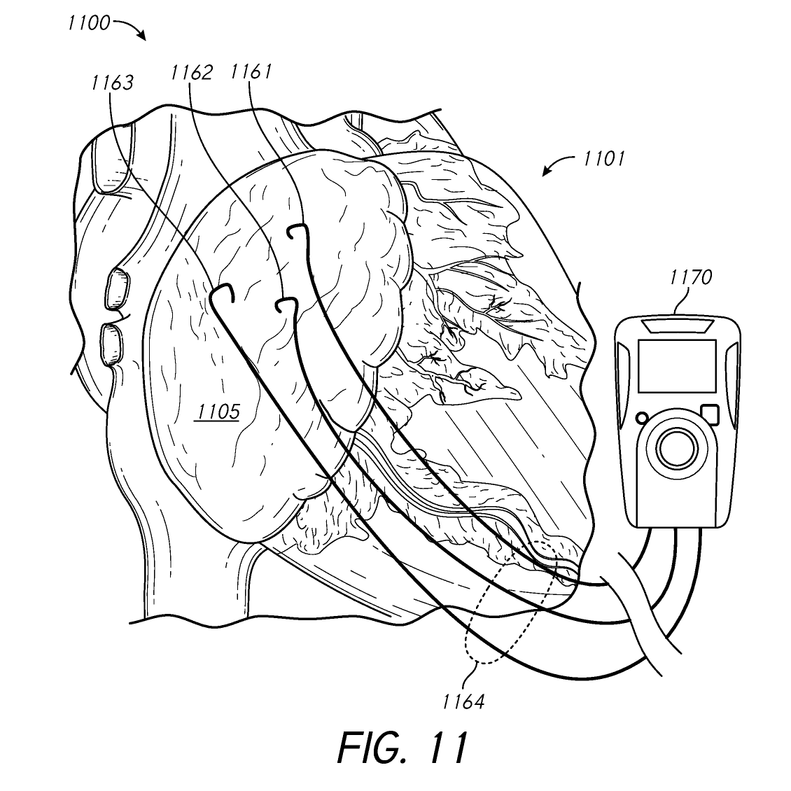

FIG. 11 illustrates a portion of a heart having disposed and/or implanted therein one or more conductive leads in accordance with one or more embodiments. As referenced above, ECG-type leads can be affixed to the external chest wall for cardiac electrical signal determination in connection with certain medical applications. However, many variables can be associated with detecting atrial conduction path disturbances using traditional manual P-wave analysis, which can lead to misdiagnosis of atrial fibrillation or delayed diagnosis of atrial fibrillation. In accordance with certain embodiments, similar to pacing leads placed in cardiac surgery, conductive leads can be placed into the atrial wall for electrical signal/waveform analysis. Such conductive leads can be constructed from insulated metallic wire with exposed tips of the wire embedded into the atrial wall. Removal of the conductive leads may be similar to pacing lead removal, as described above. The removability feature(s) of the leads 1164 provide a convenient mechanism for providing pacing, ECG measurement, and/or tissue stretching measurement functionality, while not requiring permanent implants or prolonged maintenance of implanted device(s) in the body, which can improve long-term health prospects compared to permanent or indefinite/long-term implant devices

Disclosed herein are systems, devices, and methods for detecting conduction path disturbances in biological tissue, such as in an atrium of a heart, by direct measurement within the tissue (e.g., atrial wall). In some embodiments, conductive leads are placed directly onto the atrial surface, such as in connection with an open-chest surgical procedure. The conductive leads may be used to measure discreet changes in electrical/voltage signals (e.g., waveforms) associated with atrial conduction path disturbances. Monitoring devices or systems 1170 can be used to receive detected electrical signals and determine the presence or occurrence of atrial stretching. For example, atrial stretching may be determined at least in part by measuring the change in electrical impedance or resistance between the conductive leads, or attenuation of electrical signals detected at a single lead or multiple leads. The functionality of the monitor 1170 described herein may be implemented at least in part by control circuitry of the monitor 1170.

As referenced above, directly-attached conductive leads can be used in accordance with embodiments of the present disclosure to detect a change in impedance or resistance in the atrial tissue, which may be indicative of atrial stretch or electrical disturbance. Generally, as understood by those having skill in the art, resistance relates to direct currents, while impedance relates to alternating currents. For alternating currents (e.g., high-frequency signals), inductance and capacitance in the tissue affects the impedance of the tissue. Inductance generally causes back current that reduces the overall current flowing through the tissue, whereas capacitance causes charge build-up that can reduce current. Embodiments of the present disclosure advantageously provide for determination of atrial stretch based at least in part on attenuation or change in electrical signals/waveforms, whether such attenuation/change is due to resistance or impedance. Although impedance determination is disclosed herein in connection with certain embodiments, references to impedance herein may be understood to describe or relate to impedance or resistance.

The system 1100 of FIG. 11 includes a plurality of leads 1164 attached to an atrium 1105 of a heart 1101. The leads 1164 may be placed for substantially continuous monitoring of an atrial conduction path and may serve to detect electrical signals/waveforms that are used by a monitor 1170 to detect discrete electrical disturbances and activate alarm or notification functionality to allow for intervention before the atrial tissue is permanently damaged (e.g., stretched-out), which may result in the onset of atrial fibrillation. The monitor 1170 may be configured with audible and/or visual alarm component(s) or circuitry for notifying medical personnel when conduction disturbances are detected. When informed in connection with relatively early detection of discrete disturbances to the electrical conduction path, medical personnel can modify clinical practices in order to prevent or reduce incidences of post-operative atrial fibrillation. Such modifications can include limiting or modifying intravenous fluid infusion, medication modification (e.g., diuretic medication) or intervention, and the like.

The conductive leads 1164 may be placed at positions determined to lie in electrical conduction paths of the atrium. Before a surgical operation or soon thereafter, the monitor 1170 may be configured to measure baseline voltage and/or impedance values. Such values may advantageously be stored by the monitor 1170 and identified as base-line measurements. For a period of time after surgery, the monitor 1170 may continue to measure voltage signals/waveforms, and/or determine impedance measurements (e.g., for each heart beat). Electrical signal/waveform and/or impedance measurements may be compared to the baseline values to determine whether atrial stretching has occurred. Although certain embodiments are disclosed herein in the context of impedance measurements, such description may be interpreted to refer to impedance measurements or other measurements or analysis of electrical signals/waveforms in the atrium.

The monitor 1170 may be configured to initiate an alarm indication, using one or more visual and/or audible alarm mechanism/devices, if the discrepancy between the baseline and continuous measurements exceed a predetermined set point or threshold. As referenced above, as the atrial tissue between one or more of the leads 1164 stretches, the impedance of the tissue may generally increase. In some embodiments, the monitor 1179 comprises control circuitry configured to introduce a discrete voltage signal/waveform on one or more of the leads 1164. For example, a voltage signal/waveform may be introduced into the atrial tissue using a first lead 1163, wherein the introduced signal may be received or detected by one or more additional leads, such as one or more of lead 1162 and lead 1161. The received signal/waveform may be provided by the lead(s) (e.g., 1162, 1161) to the monitor 1170, the control circuitry of which may be configured to measure impedance and/or other characteristic(s) of the signal/waveform based thereon.

In some embodiments, the monitor 1170 uses one or more of the leads 1164 to introduce an alternating current (AC) signal into the atrial tissue. The AC signal may advantageously be a high-frequency signal. Generally, the property of the tissue between the leads may determine the characteristics (e.g., time constant, attenuation, etc.) of the signal received by one or more leads. Use of high-frequency signals by the monitor 1170 may provide desirable signal fidelity at the receiver lead(s). However, signals/waveforms having any suitable or desirable frequency, amplitude, phase, or other characteristics may be used.

The leads 1164 may be spaced any suitable or desirable distance d. For example, leads may be positioned on the atrial surface approximately 1″ apart, or other distance. As the tissue stretches, the distance d may change. For example, for certain pairs of leads, the distance may increase as the atrium dilates. For example, atrial dilation/stretch may cause the distance d to increase from approximately 1″ to approximately 1.2″ in some conditions. Although certain embodiments are disclosed herein in the context of increasing distance between pairs of leads, in some embodiments, the monitor 1170 may be configured to determine atrial stretch based on increased distance between a lead and the sinoatrial (SA) node of the heart, or other electrical node. For example, a signal received on a lead may be the natural cardiac electrical signal originating at the SA node. As the atrium stretches, the tissue between the lead and the SA node may become stretched or otherwise modified, resulting in a changed signal/waveform received at the lead. Such change may indicate atrial stretch and may trigger alarm notification by the monitor 1170.

The monitor 1170 may comprise volt meter circuitry. In some embodiments, the monitor 1170 is configured to implement application of a sub-threshold high-frequency voltage and current adjustments in order to produce desired resolution. The patient monitor may be battery-powered or may be powered by standard power receptacles. In some embodiments, the monitor 1170 comprises one or more visual display devices or indicators (e.g., LEDs, LCD screen, etc.) and/or audible alarm devices. In some embodiments, the monitor 1170 is configured as a module to plug into standard patient monitors. The monitor 1170 may advantageously comprise circuitry configured to detect voltage measurements (e.g., for conduction path disturbance monitoring) between 0 to approximately 500 mV or more. With respect to impedance determination and measurement, the monitor 1170 may advantageously be configured to determine impedances between about 0-1000 Ohms.

Electrical impedance measurements can be further improved by application of a relatively low-voltage, high-frequency signal applied by the monitor 1170 to the myocardial tissue of the atrium 1105 to more accurately sense changes in impedance or other waveform characteristics. The monitor 1170 may detect changes to any characteristic of the waveforms, such as changing peak amplitude, phase, or the like. The control circuitry of the monitor 1170 comprises one or more filters or calibration features configured to implement aspects of the functionality described herein.

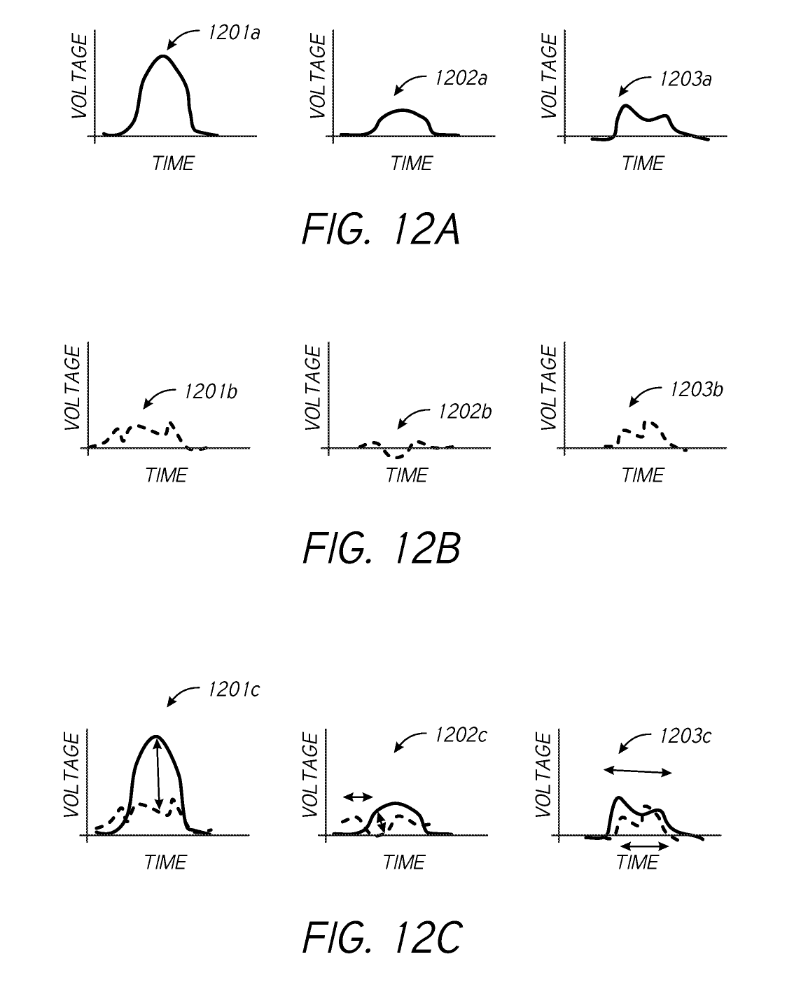

FIGS. 12A-12C illustrate example waveforms for signals propagating and/or received in atrial tissue in accordance with one or more embodiments. FIG. 12A shows a plurality of example waveforms 1201a-1203a that may be detected at respective conductive leads attached to atrial tissue, as described herein. The detected waveforms may be cardiac signals (e.g., originating in the SA node), or may be signals introduced into the atrial tissue by one lead and detected by another lead. For example, each of the waveforms 1201a-1203a may correspond to a signal transmission between respective pairs of leads of the leads 1164 shown in FIG. 11 and described above. With further reference to FIG. 11, the monitor 1170 may be configured to measure voltage signal between the leads 1164 placed in the atrial wall 1105. The waveforms 1201a-1203a may represent baseline waveforms and may be determined/collected prior to surgery or soon or immediately after surgery.

After the baseline waveform(s) (e.g., one or more of waveforms 1201a-1203a) have been determined and/or stored by the monitor 1170, the monitor may implement substantially continuous or periodic ongoing waveform determination and/or monitoring (e.g., with every cardiac cycle or period of the waveforms) for a post-operative period to detect atrial stretch and/or determine or predict the onset of post-operative atrial fibrillation. For example, atrial stretch monitoring may be performed for a period of up to 5 days after surgery, or longer.

FIG. 12B shows a plurality of example waveforms 1201b-1203b that may be detected at conductive leads attached to atrial tissue. For example, the waveforms 1201b-1203b may be detected by the respective leads associated with waveforms 1201a-1203a in FIG. 12A. Specifically, the waveforms 1201b-1203b may represent detected waveforms after conduction path disturbances have formed in the atrial tissue due to atrial stretching. Generally, when the atrial tissue becomes stretched-out, action potential curves may assume a modified shape compared to pre-stretch waveform propagation and/or detection. The waveforms 1201b-1203b may represent at least partially deformed waveforms measured a period of time after surgery, such as one or more days after surgery. By detecting/measuring waveforms at a plurality (e.g., more than two) of conductive leads can provide a relatively more complete understanding of atrial stretching in multiple directions compared to single-lead (or double-lead) implementations.

With further reference to FIG. 11, the monitor 1170 may be configured to determine differences between the baseline waveform(s) 1201a-1203a and the subsequently-detected waveform(s) 1201b-1203b. If such differences are significant, such as if measured points or values associated with the difference between the waveforms is greater than a predetermined threshold, the monitor 1170 may be configured to implement alarm or notification functionality indicating atrial stretch. FIG. 12C shows overlays of the waveforms in FIGS. 12A and 12B to illustrate differences in amplitude, phase, shape, and/or other characteristic(s) between the example waveforms that may be determined by the monitor 1170. Although waveforms are described herein corresponding to signals having a frequency component (i.e., non-DC signals), it should be understood that in some embodiments, DC voltages are transmitted, determined, and/or compared by the monitor 1170 for atrial stretch detection purposes.

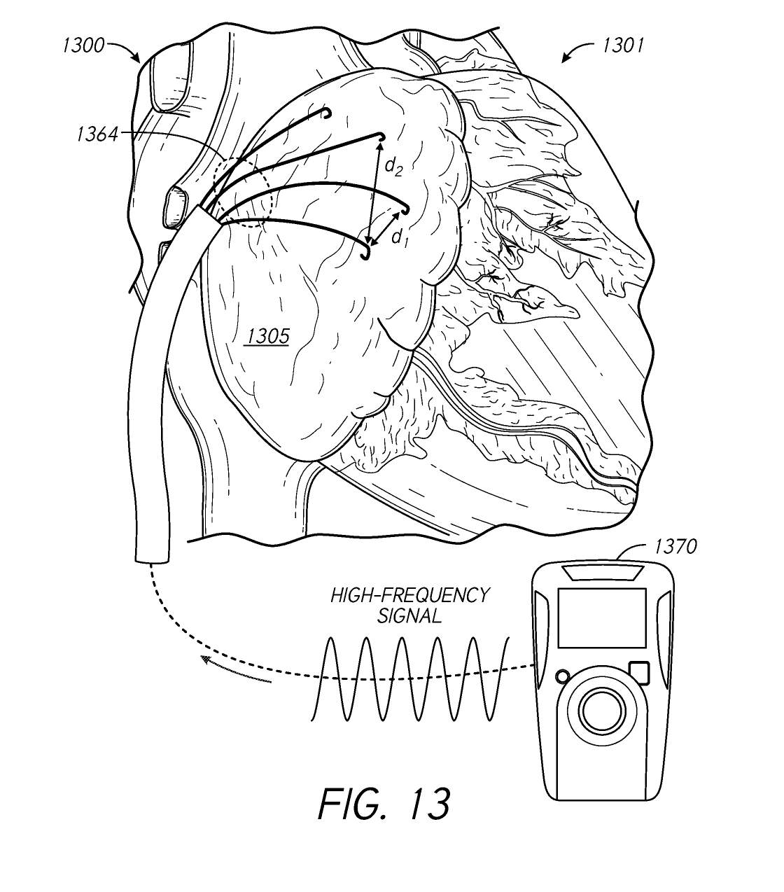

Although certain embodiments are described above in the context of detecting natural cardiac signals in atrial tissue and making atrial stretch determinations based thereon, as referenced above, in some embodiments of the present disclosure, high-frequency signals generated by a monitor system/device are introduced into atrial tissue using one or more conductive leads and detected using one or more conductive leads after propagation through at least a portion of the atrial tissue. FIG. 13 illustrates a portion of a heart 1301 having disposed and/or implanted therein one or more conductive leads 1364 in accordance with one or more embodiments. The system 1300 of FIG. 13 further comprises a monitor 1370 configured to introduce and detect high-frequency signals in the atrial tissue 1305 and make atrial stretch determinations based thereon. Such determinations may be based on waveform analysis, as described above, and/or impedance measurements (e.g., impedance of atrial myocardium) based on detected signals. Determination of electrical impedance of biological tissue is described in “Early Detection of Acute Transmural Myocardial Ischemia by the Phasic Systolic-Diastolic Changes of Local Tissue Electrical Impedance,” Jorge, E., Amoros-Figueras, G., García-Sánchez, T., Bragós, R., Rosell-Ferrer, J., and Cinca, J., Am J Physiol Heart Circ Physiol. 2016; 310: H436-H443, which is incorporated herein in its entirety.

The monitor 1370 is configured to transmit high-frequency signals 1302 into the tissue 1305 via one or more of the conductive leads 1364 for the purpose of measuring discrete conduction path variations based upon the principal that electronic coupling of cells of the myocardium of the atrial tissue may have differing impedance characteristics/values if the tissue is stretched compared to non-stretched tissue. The high-frequency signals may have a frequency between 1-1000 KHz, or greater, and may have a peak amplitude of approximately 1-mA, or greater. The detected signal may be sampled at any frequency, such as 5 MHz. Although high-frequency signals are described, in some embodiments, lower-frequency signal(s) may be employed. Micro-conductivity measurement by the monitor 1370 may provide an alternative means by which to detect conduction path disturbances relative to certain other embodiments disclosed herein. The image on the left shows a very simple 4 lead method for measuring the impedance changes within a small segment of tissue.

Although conductive leads are illustrated and described in the present disclosure as being individually and directly embedded in atrial tissue, it should be understood that conductive leads in accordance with the present disclosure may be electrically coupled to the atrial tissue in any suitable or desirable manner or using any type of attachment/connection means. For example, in some embodiments, one or more leads are integrated with a printed flex circuitry. Such a printed flex circuit may advantageously be used in connection with a pull wire release mechanism or other release mechanism as described herein. Printed flex circuit lead coupling structures in accordance with the present disclosure may comprise one or more of a thin plastic printable circuit that is configured to be affixed to atrial tissue and provide an electrical interface between one or more exposed conductive leads and the contacting tissue. In some embodiments, the flex circuit is attached to the atrial surface using one or more sutures, which may be bioresorbable or coupled to the circuit using pull-wire component(s).

In some embodiments, conductive leads are electrically coupled to the atrial surface using a bioresorbable membrane having bioinert conductive ink tracing (e.g., iron, magnesium, or the like). The membrane may be maintained affixed to the atrium for a post-operative period. In some embodiments, the bioresorbable membrane comprises polyester, or the like. Rather than copper or other conductive wire, the distal portion of the conductive lead(s) can comprise magnesium or other type of wire that can break-down over time. In some embodiments, conductive ink is implemented for at least a portion of the conductive lead(s), which may comprise fine metal powder suspended in a polymer binder, or other material or configuration. The flexible membrane may house the distal portions of the conductive lead(s) such that they are evenly spaced and easily inserted/coupled into the tissue together.

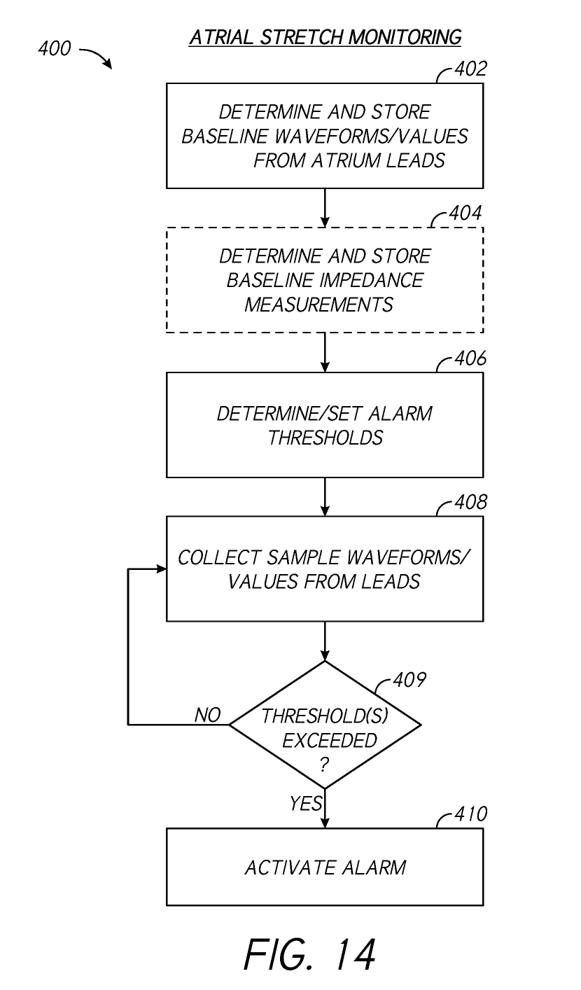

FIG. 14 is a flow diagram illustrating a process 400 for monitoring stretching in biological tissue in accordance with one or more embodiments of the present disclosure. At block 402, the process 400 involves determining, characterizing, and/or storing baseline waveforms and/or associated values (e.g., voltage values) detected or determined using one or more conductive leads embedded or otherwise attached to the atrium of a heart. Baseline waveform/value characterization may be performed using readings from a plurality of conductive leads, such as three or four leads, and may involve identifying one or more waveform characteristics and/or associated values, such as P-wave duration, amplitude area under the waveform, distance to Q wave, and the like. Use of multiple leads for detecting electrical signals may advantageously provide improved fidelity and/or redundancy for waveform analysis purposes. In embodiments utilizing a single conductive lead, it may be desirable for a grounding reference structure or pad to be electrically coupled to a portion of the patient's body.

At block 404, the process 400 optionally involves determining, characterizing, and/or storing baseline impedance measurements based on the baseline waveforms or values determined at block 402. In some embodiments, impedance may be calculated based on baseline voltage and/or current values associated with a source of the detected electrical signal(s). For example, the source may be a natural electrical signal of the heart, such as may be generated at the sinoatrial node of the heart, or the signal may be an artificially-generated signal, which may be introduced into the atrial tissue using one or more conductive leads, as described herein. That is, impedance measurements/determinations may represent impedance between two separate conductive leads, or impedance between a conductive lead and a natural cardiac electrical signal source. For example, as referenced above, natural cardiac signals are generally generated in myocardial tissue of the heart to make the heart muscles contract. Impedance determinations/calculations may be affected at least in part by characteristics of the biological tissue, including the presence of fatty tissue, blood vessel(s), and/or other features. Furthermore, where one or more of the conductive leads becomes at least partially dislodged or altered, such issue may affect impedance in a way that is not necessarily related to, or caused by, tissue stretching. Therefore, care may advantageously be taken to ensure proper or desired contact between the lead(s) and the atrial tissue is maintained.

At block 406, the process 400 involves determining and/or setting alarm threshold values. Such threshold values may be related to impedance values, voltage values, and/or other waveforms or signal characteristics, including waveform shape-related values, or the like., Such predetermined threshold values may be stored in a monitoring device using control circuitry thereof. For example, P-waveforms may be detected/collected from a plurality of leads, wherein control circuitry of the monitor device or system is implemented to characterize one or more aspects or features of the waveforms (e.g., P-wave duration, amplitude, area under the curve, distance to Q wave, and the like).

At block 408, the process 400 involves detecting or determining or detecting additional sample waveforms or other signal values using one or more conductive leads embedded or otherwise electrically coupled to the atrial tissue. Such collection or detection may be performed on an ongoing basis after a surgical operation for a post-operative period, such as one or more days, or longer. That is, while the determination/characterization of blocks 402 and/or 404 may be performed before surgery or immediately afterwards, the determination/detection of waveforms/values at block 408 may be performed to track changes in waveforms/values and/or impedance over time during a post-operative period to detect or predict instances of atrial stretching and/or atrial fibrillation.

At decision block 409, the process 400 involves determining whether the detected/collected sample waveforms/values exceed the predetermined threshold levels associated with block 406 and described above. For example, the process may involve determining differences in values or characteristics of signals received and/or provided on different leads. In some embodiments, multiple waveforms are analyzed, whether associated with natural electrical signals or induced/introduced electrical signals. The determination of block 409 may involve measuring differential or absolute values. In some embodiments, the determination is made at least in part by subtracting an area under a waveform curve from associated baseline waveform data. When the shape of a waveform changes to a significant degree, such change may indicate impending fluid volume overload.

If the threshold(s) have not been met at block 409, the process 400 loops back to block 408, where additional waveforms and/or values are detected on an ongoing basis. If detected waveforms/values exceed the predetermined threshold(s), the process 400 proceeds to block 410, where certain alarm functionality may be activated or initiated in order to provide notification of atrial stretching, as described in detail herein. In embodiments employing multiple conductive leads, voltage/waveform measurements may indicate directionality of atrial stretch and/or allow for detection of stretch in multiple directions. Furthermore, depending on which lead certain waveforms/values are detected on, the detected data can indicate a location of stretch. Such information may be communicated in connection with the alarm/notification step 410. The process 400 may be performed at least in part by control circuitry of a monitoring system or device of any of the disclosed embodiments and configured to implement certain functionality disclosed herein. Although a certain order is illustrated in FIG. 14 and described above in connection therewith, it should be understood that the steps or operations associated with the process 400 may be performed or executed in any suitable or desirable manner. In addition, certain of the illustrated steps of the process 400 may be omitted in some embodiments and/or additional steps not illustrated or described explicitly may be included within the scope of the present disclosure.

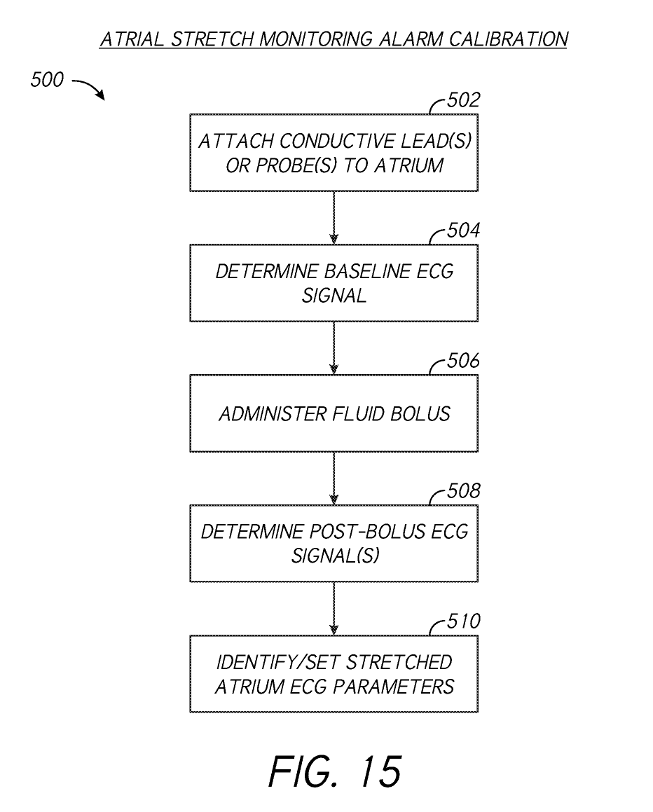

FIG. 15 illustrates a flow diagram 500 for calibrating an alarm threshold for atrial stretch in accordance with one or more embodiments. The process 500 may be implemented to determine one or more alarm setpoints/thresholds for triggering an alarm or notification in connection with stretch-detection devices or methods disclosed herein. The process 500 may be implemented in connection with a patient having one or more conductive leads embedded or otherwise attached to the atrium of a heart of the patient. Furthermore, the process 500 may be implemented in connection with one or more direct-implant ECG probes, as shown in FIGS. 5-9 and described above, which may provide electrical signals relating to the heart's cardiac system.

At block 502, the process 500 involves attaching, implanting, or otherwise electrically coupling one or more conductive leads or probes in accordance with embodiments of the present disclosure to the atrium of a patient's heart, thereby electrically coupling a monitor device or system to the atrium, as described in detail herein. At block 504, the process 500 involves determining and/or inputting a baseline cardiac signal determined using the direct-implanted lead(s)/probe(s).

Generally, when conductive leads are attached to the atrium or other internal cardiac tissue of the patient, there may be access to a central venous line of the patient, which may allow for relatively convenient introduction of intravenous (IV) fluid into the patient. At block 506, the process 500 involves administering a bolus of fluid, such as saline fluid or the like, into the vascular system of the patient. Such bolus may be any suitable or desirable volume, such as hundred milliliters, or other volume bolus. Administration of the bolus at block 506 may be performed after a surgical operation in some embodiments.

At block 508, the process 500 involves determining and/or inputting post-bolus ECG signals of the patient using the direct-implanted lead(s)/probe(s). Such post-bolus ECG measurements may advantageously have parameters associated therewith indicating P-wave disturbance or other attributes of the ECG signal associated with atrial stretching and/or fluid overload.

At block 508, the process 500 involves determining, identifying, and/or setting stretched-atrium ECG parameters indicated by the post-bolus ECG measurement(s) as being associated with atrium stretching. Additionally or alternatively, the ECG parameters may relate to impedance values, such as changes in impedance values. The parameters identified as being associated with post-bolus atrium stretching may be used to set alarm thresholds for the monitor device/system, wherein identification of such parameters in subsequently collected/determined post-operative ECG signals can be used to trigger alarm functionality.

### ADDITIONAL EMBODIMENTS

Depending on the embodiment, certain acts, events, or functions of any of the processes described herein can be performed in a different sequence, may be added, merged, or left out altogether. Thus, in certain embodiments, not all described acts or events are necessary for the practice of the processes. Moreover, in certain embodiments, acts or events may be performed concurrently.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A direct-implantable electrocardiographic (ECG) probe device comprising:
a biocompatible housing;
a battery disposed within the housing;
one or more electrodes including an ECG electrode configured to sense an electrical signal in tissue of an atrium of a heart;
circuitry disposed at least partially within the housing and configured to generate an ECG signal and wirelessly transmit the ECG signal through a chest wall; and
an attachment structure configured to facilitate attachment of the ECG probe device to a surface of the atrium.

2. The direct-implantable ECG probe device of claim 1, wherein the attachment structure comprises one or more suture holes.

3. The direct-implantable ECG probe device of claim 1, wherein the attachment structure comprises a pin form configured to puncture the surface of the atrium.

4. The direct-implantable ECG probe device of claim 1, further comprising a grounding structure.

5. The direct-implantable ECG probe device of claim 4, wherein the grounding structure is disposed on an underside of the housing and configured to contact the surface of the atrium when the ECG probe device is implanted on the surface of the atrium.

6. The direct-implantable ECG probe device of claim 1, wherein the one or more electrodes includes a pacing electrode configured to introduce a jolt of electrical current to the surface of the atrium.

7. The direct-implantable ECG probe device of claim 6, wherein the pacing electrode and the ECG electrode are the same.

8. The direct-implantable ECG probe device of claim 1, wherein housing is at least partially disk-shaped.

9. A heart monitoring system comprising:
a plurality of electrocardiographic (ECG) leads configured to:
be directly implanted in a surface of an atrium of a heart of a patient;
sense an electrical signal in tissue of the atrium; and
provide an ECG signal based on the sensed electrical signal;
a monitor device coupled to the ECG leads and configured to receive the ECG signal; and
a grounding pad electrically coupled to the monitor device.

10. The heart monitoring system of claim 9, wherein the monitor device is configured to identify a change in one or more P-wave characteristics in the ECG signal associated with atrial fibrillation.

11. The heart monitoring system of claim 10, wherein the monitor device is further configured to generate an alarm notification based on said identification of the change in the one or more P-wave characteristics.

12. The heart monitoring system of claim 9, further comprising a plurality of pacing leads configured to be directly implanted in the surface of the atrium, the plurality of pacing leads being coupled to the monitor device.

13. The heart monitoring system of claim 12, wherein the monitor device is configured to present an electrical charge on one or more of the pacing leads in response to the ECG signal.

14. A method of generating an electrocardiographic (ECG) signal, the method comprising:
implanting one or more ECG probes on a surface of a heart of a patient; and
generating an ECG signal using the implanted one or more ECG probe devices.

15. The method of claim 14, wherein the one or more ECG probes are discrete implantable devices.

16. The method of claim 15, further comprising wirelessly receiving the ECG signal from the one or more ECG probes through a chest wall of the patient.

17. The method of claim 14, wherein the one or more ECG probes are wire leads.

18. The method of claim 17, further comprising disposing the wire leads in a chest-access channel in a chest of the patient.

19. The method of claim 14, further comprising implanting one or more pacing leads in the surface of the heart.

20. The method of claim 19, further comprising delivering a dose of electrical current to the heart using the one or more pacing leads.

21. The method of claim 14, further comprising closing a chest cavity of the patient after said implanting the one or more ECG probes and before said generating the ECG signal.

22. The method of claim 14, further comprising identifying a characteristic in the ECG signal that is associated with atrial fibrillation.

23. The method of claim 14, further comprising determining an impedance associated with a portion of the heart based at least in part on the ECG signal.
